(19) United States
(12) Patent Application Publication
(10) Pub. No.: US 2014/0081145 A1
(43) Pub. Date: Mar. 20, 2014

(54) MULTI-CHANNEL HEMISPHERICAL ULTRASONIC WAVE TRANSMIT AND RECEIVING DETECTION SYSTEM AND METHOD

(71) Applicant: Chang Gung University, Tao-Yuan (TW)

(72) Inventors: Hao-Li LIU, Tao-Yuan (TW); Chen-Kai JAN, Guishan Township (TW); Guan-Lu HUANG, New Taipei City (TW); Hao-Yu JHONG, Luzhu Township (TW)

(73) Assignee: Chang Gung University, Tao-Yuan (TW)

(21) Appl. No.: 14/089,227

(22) Filed: Nov. 25, 2013

Related U.S. Application Data
(62) Division of application No. 13/450,213, filed on Apr. 18, 2012.

(30) Foreign Application Priority Data
Apr. 19, 2011 (TW) 100113578

(51) Int. Cl. A61B 8/08; A61B 8/00

(52) U.S. Cl. CPC A61B 8/085; A61B 8/461; A61B 8/4488; A61B 8/467; A61B 8/56; A61B 8/4455
USPC 600/459; 600/437

(57) ABSTRACT

A multi-channel hemispherical ultrasonic wave transmit and receive detection system and method, wherein, a transmit control system transmits out a plurality of detection signals through an energy converter, a receive system receives the reflected signals reflected from an obstacle, in determining a channel used by said transmit control system to control a multiplexer to select a channel to switch to and then amplify said reflected signals. Then, said receive system performs high speed analog-to-digital sampling, and high speed transmission through USB for said reflected signals amplified. Finally, a Graphic User Interface (GUI) of a computer performs smooth-out processing for said reflected signals and displays its waveforms, in determining distance to said obstacle.

### CROSS REFERENCE TO RELATED APPLICATIONS

This application is a Divisional of co-pending application Ser. No. 13/450,213, filed on Apr. 18, 2012, for which priority is claimed under 35 U.S.C. §120; and this application claims priority of Application No. 100113578 filed in Taiwan, R.O.C. on Apr. 19, 2011 under 35 U.S.C. §119; the entire contents of all of which are hereby incorporated by reference.

### BACKGROUND OF THE INVENTION

1. Field of the Invention

The present invention relates to a technology of ultrasonic wave detection, and in particular to a multi-channel hemispherical ultrasonic wave transmit and receive detection system and method embed in a hemispherical-type ultrasound phased-array.

2. The Prior Arts

The application of ultrasonic wave in medication can be classified into a diagnostic application, and a therapy application. Wherein, energy of ultrasonic wave for diagnostic application is weaker, such as ultrasonic wave imaging used for rib detection; while energy of ultrasonic wave for therapy application is stronger, such as focusing type ultrasonic wave.

Presently, the focusing type ultrasonic wave has been proved to be effective in non-intrusion type liver tumor heat burning therapy. However, rib will cause blocking of ultrasonic wave energy, and thus defocusing of energy, and the problem of overheating of ribs. Therefore, rib blocking is a rather large obstacle in therapy process. Due to the problem of ripple waves generated by the piezoelectric plate and transmit system, thus in signal determination, the location of burr signal can not be ascertained, and all that is known is that, signals are moving on the ripple wave, therefore, subsequent analyses may bring quite a lot of misjudgment.

Focused ultrasound thermal ablation technology first appeared half a century ago (Lynn et al 1942), and has recently received increasing interest (Chapelon et al 1991, Hynynen et al 1993, Sanghvi and Hawes 1994, ter Haar 1995). The unique characteristic of focused ultrasound is that the energy can be focused onto soft tissues noninvasively and induce a localized temperature elevation (30-55° C.) within a few seconds. The resulting high temperature can generate irreversible tissue necrosis at the target region while not damaging the surrounding normal tissues.

Recent clinical studies have shown the feasibility, safety, and effectiveness of the focused ultrasound treatment modality for treating hepatocellular carcinoma (HCC) and other liver tumors. (Wu et al 2004a, Wu et al 2004b)) demonstrated the safety and efficacy of focused ultrasound treatment in over 50 patients with HCC (tumor size ranged from 4 to 14 cm) using a 0.8-MHz, spherical single-element focused ultrasound transducer (JC-type, Chongqing HIFU Technology, Chongqing, China). (Illing et al 2005) and (Kennedy et al 2004) found that the same treatment unit was feasible and allowed noninvasive access to the tumor in 27 liver-cancer patients. However, some complications still need to be considered, For example, skin burns accompanying the treatments need to be resolved: third-degree skin burns can be induced (Wu et al 2004b), which are usually associated with the ribs overlying the treatment region. These ribs can attenuate the transmission of ultrasound energy toward the target area, and also absorb or reflect the incident energy so as to cause skin burns. Moreover, the ribs represent a large inhomogeneity in the medium that can cause phase aberrations, with the resulting focal beam distortions (Liu et al 2005) preventing the required temperature increase at the target area. These problems have lead to a requirement for the partial surgical removal of ribs prior to a focused ultrasound ablation session, which significantly degrades the noninvasive nature of focused ultrasound therapy (Illing et al 2005, Wu et al 2004b).

Improving focused ultrasound thermal ablation for liver treatment requires a comprehensive investigation of its characteristics so as to provide an optimal treatment configuration that includes the prevention of skin burns and the avoidance of rib surgery. Overcoming these obstacles may be possible with an ultrasound phased array, which can provide more flexible and dynamic focusing than a spherical single-element transducer. Moreover, the element of the phased array can be independently controlled to be activated or turned off. Advantages above increase the possibility to treat the liver tumor through intact ribs.

U.S. Pat. No. 6,735,461 discloses that the magnetic resonance imaging (MRI) can be synchronize with the focused ultrasound apparatus and operate simultaneously, and maybe have some potential to explore the therapeutic application such as the U.S. Pat. No. 5,752,515. U.S. Pat. No. 6,785,572 and U.S. Pat. No. 6,064,904 also discloses a system for conducting a computed tomography (CT) imaging-guided medical procedure on a subject includes a medical imaging apparatus which can obtain medical images for treatment target localization. It is, therefore, possible to develop a real-time ultrasound transmit/received based technology for identifying the precise locations of the chest ribs and then for the utilization of the focused ultrasound therapy, so that the real-time dynamic ultrasound element activation/deactivation selection can be realized to overcome the repertory body motions during treatment.

Therefore, presently, the design and application of ultrasonic wave detection system and method of the prior art are not quite satisfactory, and it has much room for improvements.

### SUMMARY OF THE INVENTION

In view of the problems and shortcomings of the prior art, the present invention provides a multi-channel hemispherical ultrasonic wave transmit and receive detection system and method, that can solve, the problem of the prior art.

A major objective of the present invention is to provide a multi-channel hemispherical ultrasonic wave transmit and receive detection system and method on hemispherical-type ultrasound phased array, that receives the reflected signals of the ultrasonic wave detection signals transmitted, and then performs high speed sampling of the reflected signals, and transmits the sampled signal to a Graphic User Interface (GUI) at high speed through USB, to calculate the position of the reflected signal (namely, position of the ribs).

Another objective of the present invention is to provide a multi-channel hemispherical ultrasonic wave transmit and receive detection system and method, which utilizes the principle of transmitting energy through ultrasonic wave, to receive energy of the reflected signals, for displaying it on a Graphic User Interface (GUI) without causing any deviations.

In order to achieve the above-mentioned objective, the present invention provide a multi-channel hemispherical ultrasonic wave transmit and receive detection system, comprising: a transmit control system; a multiplexer; a receive system; and a Graphic User Interface (GUI). Wherein, the transmit control system sends out detection signals of a plurality of single channels; the multiplexer is used to switch channels, and to amplify the detection signals and the reflected signals of ultrasonic wave that can not transmit through an obstacle; the receive system determines the channel of the transmit control system used for the detection signals, receives the reflected signals amplified, and performs high speed analog-to-digital sampling for the reflected signals amplified, and transmits them at high speed to a GUI through a USB; and the Graphic User Interface (GUI) receives the reflected signals sent from the receive system through a USB communication module, performs smoothing-out of the reflected signals and displays the waveforms of the reflected signals, in determining distance to the obstacle.

Moreover, the present invention provide a multi-channel hemispherical ultrasonic wave transmit and receive detection method, comprising the following steps: sending out detection signals of a plurality of single channels by means of a transmit control system; utilizing a receive system to receive through a multiplexer the reflected signals of the detection signals that can not transmit through the obstacle; determining by the receive system the channel of the transmit control system used for the detection signal, controlling the multiplexer to switch channels, and amplifying the detection signals and the reflected signal; performing high speed analog-to-digital sampling of the reflected signal amplified by the receive system, and transmitting them to a GUI through a USB communication module; and using a GUI to display the wave forms of the reflected signals sent from the receive system via the USB communication module.

Further scope of the applicability of the present invention will become apparent from the detailed description given hereinafter. However, it should be understood that the detailed description and specific examples, while indicating preferred embodiments of the present invention, are given by way of illustration only, since various changes and modifications within the spirit and scope of the present invention will become apparent to those skilled in the art from this detailed description.

### BRIEF DESCRIPTION OF THE DRAWINGS

The related drawings in connection with the detailed description of the present invention to be made later are described briefly as follows, in which:

FIG. 1 is a schematic diagram of a multi-channel hemispherical ultrasonic wave transmit and receive detection system sending out multi-channel detection signals to a target point, and receives reflected signals from the target point;

FIG. 2 is a block diagram of a multi-channel hemispherical ultrasonic wave transmit and receive detection system according to the present invention;

FIG. 3 is a flowchart of the steps of a multi-channel hemispherical ultrasonic wave transmit and receive detection method according to the present invention;

FIG. 4 is a block diagram of a multi-channel receive system of a multi-channel hemispherical ultrasonic wave transmit and receive detection system according to the present invention;

FIG. 5 is a flowchart of the steps of a process for the reflected signal to go through analog-to-digital conversion and then be sent to GUI according to the present invention;

FIGS. 6A and 6B are waveforms of reflected signals on GUI respectively having rib blocking and without rib blocking according to the present invention; and

FIGS. 7(a) to (f) are comparison graphs of hydrophone simulation standard results and actual experiment results using rib pitches 1:2 and 1:1 according to the present invention.

### DETAILED DESCRIPTION OF THE PREFERRED EMBODIMENT

The purpose, construction, features, functions and advantages of the present invention can be appreciated and understood more thoroughly through the following detailed description with reference to the attached drawings. And, in the following, various embodiments are described in explaining the technical characteristics of the present invention.

The present invention provides a multi-channel hemispherical ultrasonic wave transmit and receive detection system and method, wherein, the system sends out detection signals respectively through the respective single channels, and a transmit control system informs the receive system to receive signals for any of its channels, and then switches the respective channels to receive all the reflected signals, then determines if the detection signals sent out are blocked by the bones.

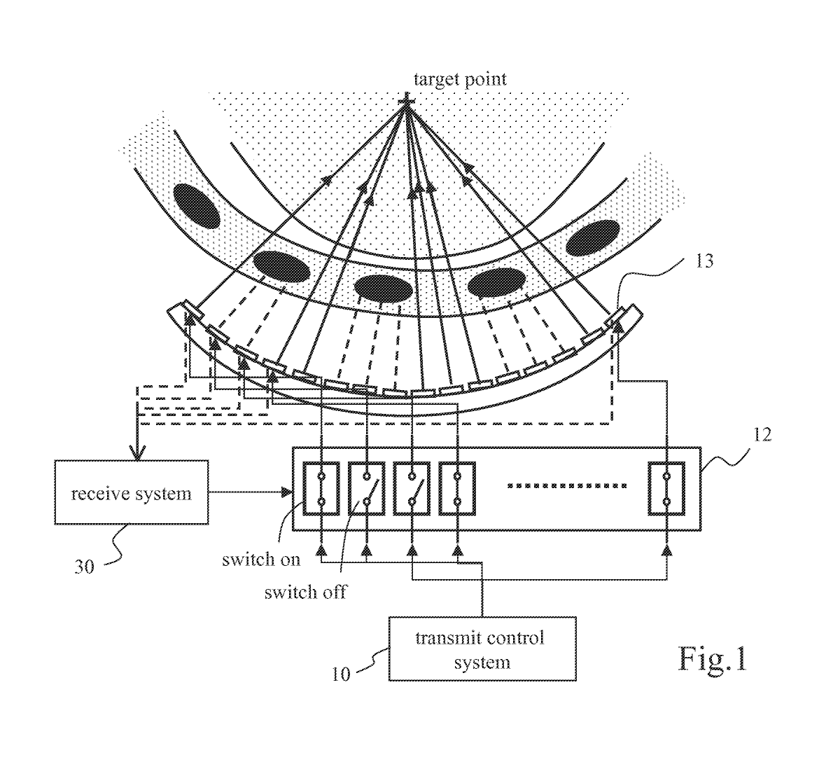

Refer to FIG. 1 for a schematic diagram of a multi-channel hemispherical ultrasonic wave transmit and receive detection system sending out multi-channel detection. signals to a target point, and receives reflected signals from the target point. As shown in FIG. 1, a transmit control system 10 sends out command to transmit ultrasonic wave signal, a signal amplification and driving system 12 receives the command, amplifies the signal and outputs it to an ultrasonic wave phase array 13, which is provided with a plurality of ultrasonic wave piezoelectric plates, each of the plate receiving the command will transmit the ultrasonic wave signal to a same target point, however, if the signal encounters an object such as a rib, and it can not transmit through, then it will be reflected back, for the same or adjacent ultrasonic wave piezoelectric plate to receive the reflected signal, and transmits it to the multi-channel receive system 30, such that a part of the signal amplification and driving system 12 is turned on, and a part of the signal amplification and driving system 12 is turned off, as shown in FIG. 1.

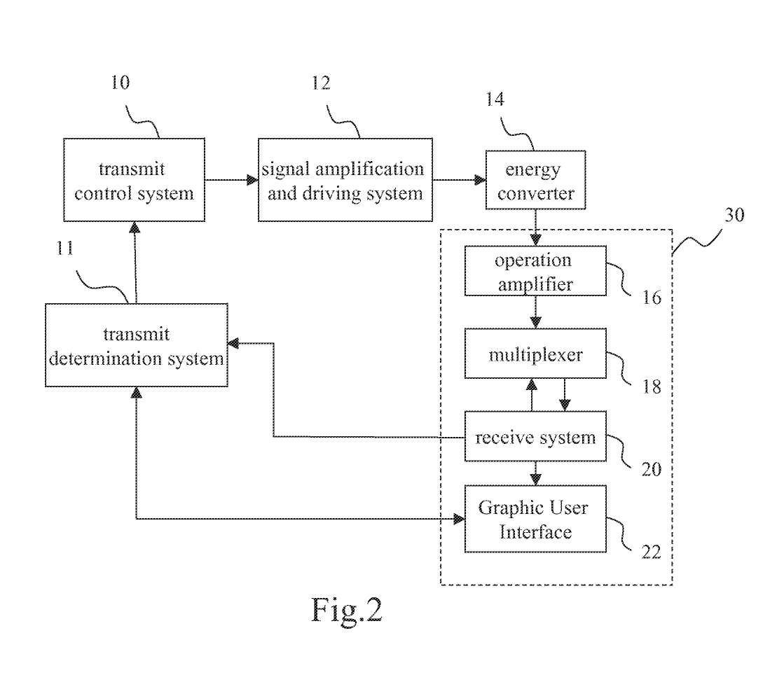

Refer to FIG. 2 for a block diagram of a multi-channel hemispherical ultrasonic wave transmit and receive detection system according to the present invention. As shown in FIG. 2, the detection system includes: a transmit control system 10, a transmit determination system 11, a signal amplification and driving system 12, an energy converter 14, and a multi-channel receive system 30. Wherein, the multi-channel receive system 30 further includes: an operation amplifier 16, a multiplexer 18, a receive system 20, and a Graphic User Interface (GUI) 22. The transmit control system 10 is the core of the overall signal control, for controlling system to system communications; the signal amplification and driving system 12 mainly functions to amplify the detection signals sent out from the transmit control system 10, to raise the signal to a voltage of over 50V, and send it out through energy converter 14. In addition, on the receiving side, the receive system 20 will receive the reflected signals to determine the channel used by the detection signal, to control the multiplexer 18 to select and switch to that channel and will amplify the signal; then the receive system 20 performs high speed analog-to-digital sampling for the reflected signal amplified, and high speed transmission to a GUI via a USB communication module. Finally, the Graphic User Interface (GUI) 22 of a computer displays waveforms of the reflected signal, and smooth out the burred waveform. Moreover, the receive system 20 further transmits the reflected signal amplified to the transmit determination system 11, in determining the distance to the obstacle based on the waveform of the reflected signal.

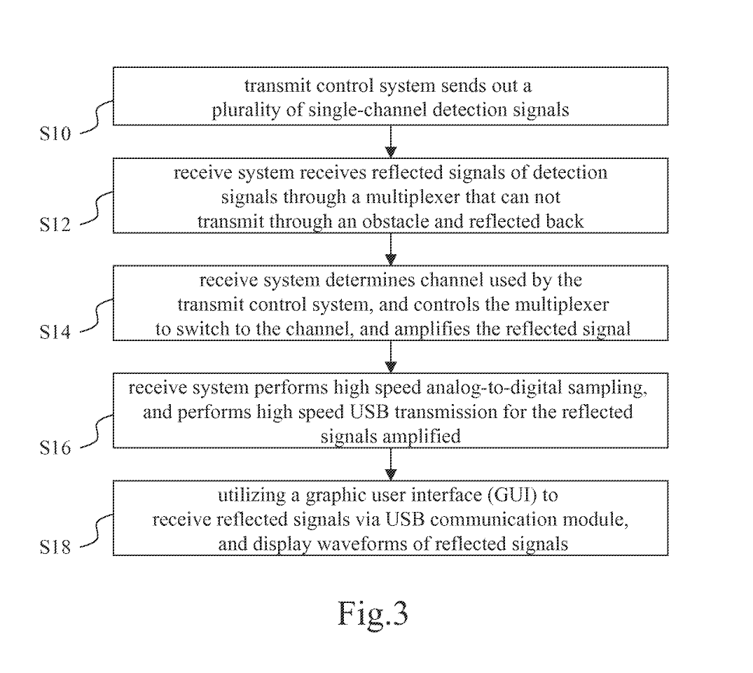

Then, refer to FIG. 3 for a flowchart of the steps of a multi-channel hemispherical ultrasonic wave transmit and receive detection method according to the present invention. As shown in FIG. 3, firstly, as shown in step S10, the transmit control system sends out a plurality of single channel detection signals, and the system is triggered by using burst waves of 5-period, such that it selects a channel to output the burst waves, meanwhile, it triggers the USB communication module with the burst waves; next, upon sending out the detection signals, part of them can not transmit through the obstacle such as a rib are reflected back, which in step S12 are received by an hemispherical ultrasonic wave probe, then the reflected signals are transmitted to a multiplexer in the multi-channel receive system, then in step S14 the multi-channel receive system determines the channel which the transmit control system utilizes to transmit the detection signal, and controls the multiplexer to switch to that channel, and it also controls the operation amplifier to amplify the detection signal and the reflected signal; subsequently, the receive system in the multi-channel receive system performs high speed analog-to-digital sampling of the reflected signal amplified, and high speed transmission of the signal to the USB communication module, as shown in step S16; then in step S18, it transmits the reflected signal to the Graphic User Interface (GUI) via the USB communication module, and after analyzing the reflected signals, the GUI displays the waveforms of the reflected signals.

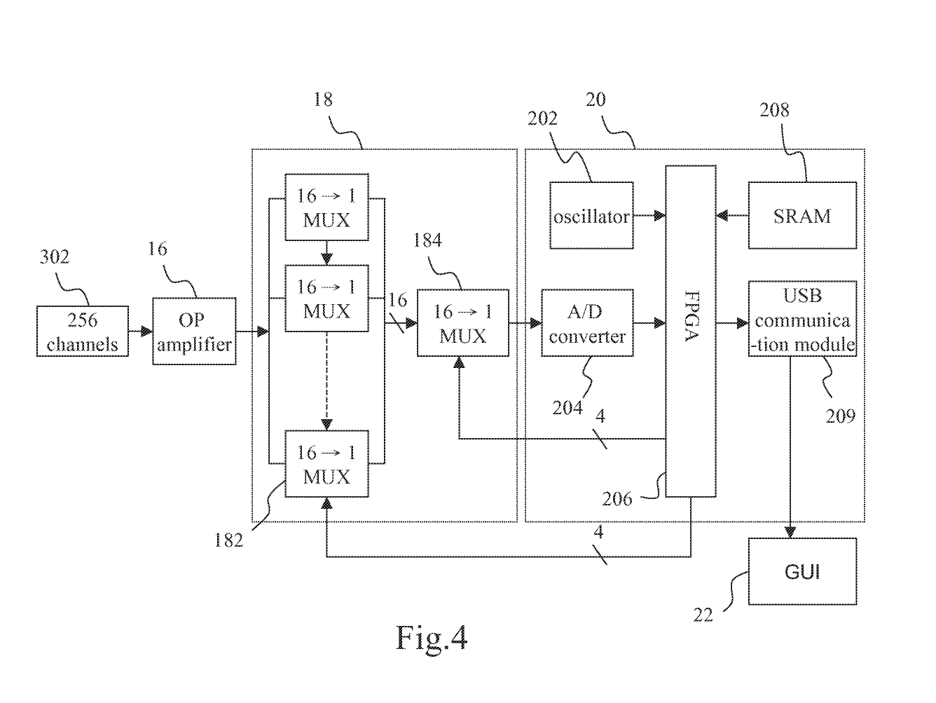

Subsequently, refer to FIG. 4 for a block diagram of a multi-channel receive system in a multi-channel hemispherical ultrasonic wave transmit and receive detection system according to the present invention. As shown in FIG. 4, the multiplexer 18 further includes 16 first stage multiplexers 182 and a second stage multiplexer 184, and all these multiplexers 182 and 184 are of 16:1 multiplexers. The receive system 20 further includes an oscillator 202, an analog-to-digital converter 204, a field programmable gate array (FPGA) 206, a static random access memory (SRAM) 208, and a USB communication module 209. The oscillator 202 is used to send out pulse signal to the field programmable gate array (FPGA) 206, to achieve overall timing correctness; the analog-to-digital converter 204 is provided With a sampling frequency of 10 MHz; the hemispherical ultrasonic wave probe receives reflected signals via one of the 256 channels 302, then the weak reflected signals are amplified by an operation amplifier 16, to avoid distortion for being too weak after they pass through the multiplexer 18 and analog-to-digital converter 204; and the static random access memory (SRAM) 208 is used to record and store continuously the reflected signals passing through the analog-to-digital converter 204 for a certain period of time.

In the present invention, the transmit control system sends out the respective detection signals via the respective 256 channels in sequence, and the reflected signals are received at the most adjacent channel in the 256 channels 302 by the multi-channel receive system. Due to the enormous amount of 256 channels, in order to save cost, in the present invention, the operation amplifier 16 is connected directly to 16 first stage multiplexers 182 of 16:1, and then they are connected to a second stage multiplexer 184 of 16:1, so one of 256 channels is selected to receive the reflected signals. The advantage of this design is that only one clamping circuit is required, hereby reducing clamping area and component cost of large amount 255 channels.

Upon receiving the reflected signals by the hemispherical ultrasonic wave probe, the reflected signals passing through the analog-to-digital converter 204 are recorded by the static random access memory (SRAM) 208 for a certain period of time, until sufficient amount of reflected signals are stored, then the transmit determination system 11 will proceed with rear end analysis, in determining the distance between the detection device and the obstacle.

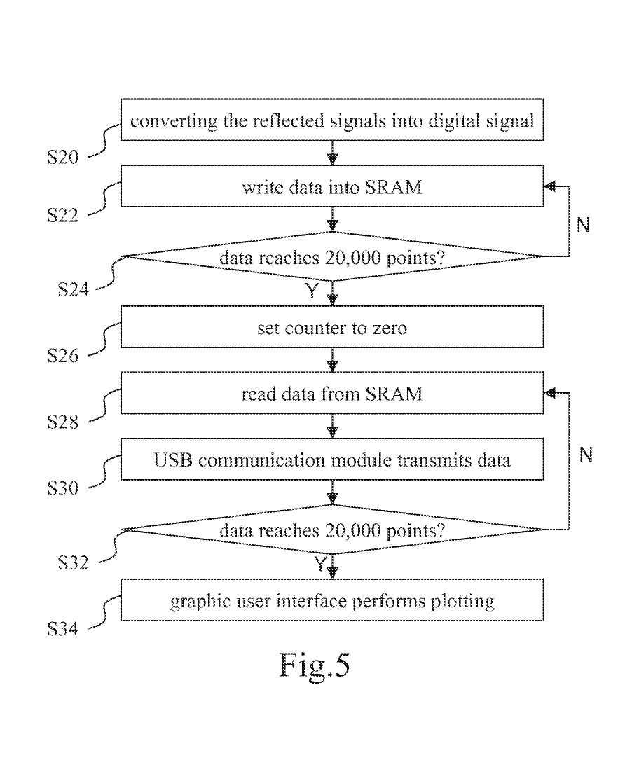

In the present invention, in order to realize plotting the graphs on GUI in synchronization with receiving the reflected signals, so after the static random access memory (SRAM) 208 receives and stores the reflected signals for a certain period of time, the receive system 20 will partition the data of the reflected signals, so as to make the field programmable gate array (FPGA) 206 to transmit 20,000 points of data into a program of GUI 22, meanwhile, the computer program will set a counter to count the points of data received, such that in case the amount of data has reached a preset value, then set the value of the counter to zero, such that FPGA will read in data from SRAM 208, and output them to GUI 22 via USB communication module 209. In the present embodiment, the case of data points of 20,000 is taken as an example, refer to FIG. 5 for a flowchart of the steps of a process for the reflected signals to go through analog-to-digital conversion and then be transmitted to GUI according to the present invention. As shown in FIG. 5, firstly, in step S20, the reflected signal are converted into digital signals after going through analog-to-digital conversion; next, in step S22, data of the reflected signal are partitioned and are written into SRAM; then, in step S24, determining if the number of data points has reached 20,000, in case the answer is negative, then return to step S22, otherwise, as shown in steps S26 to S30, set the number of counter to zero, and field programmable gate array (FPGA) 206 reads the data from SRAM, and sends the 20,000 points of data to program in the transmit determination system 11 via the USB transmission module.

Subsequently, the transmit determination system 11 determines if the number of data has reached 20,000 points, as shown in step S32, in case the answer is negative, then the process returns to step S28 to read the data again, otherwise, as shown in step S34, the transmit determination system 11 sets an X axis in a Picture Box, and places data received from USB communication module into an array, and sets a Y axis in the Picture Box, to plot the waveforms of the reflected signals in the Picture Box. Also, in order to avoid error in receiving the reflected signals, the program. will determine end of data based on 500 points of 0 appearing at the end of 20,000 points of data, thus this will not affect subsequent analyses of data. Also, since the data is arranged according to the counter, thus this approach is able to store the data of the graphs displayed in the Picture Box, and it will not overwrite the previous data.

Upon receiving the partitioned data of the reflected signals, GUI will first perform smoothing-out of the signal. Since upon reception, the reflected signal is affected by ripples and spikes, therefore sometimes it is difficult to determine existence of signal having burrs, thus leading to misjudgment in subsequent analyses. In the smooth-out process, a rated level 127 is placed between the first two data points of the signal, then taking an average value every five data points, as such removing the burst waves appearing suddenly on the waveform, so as to make the waveform look smoother.

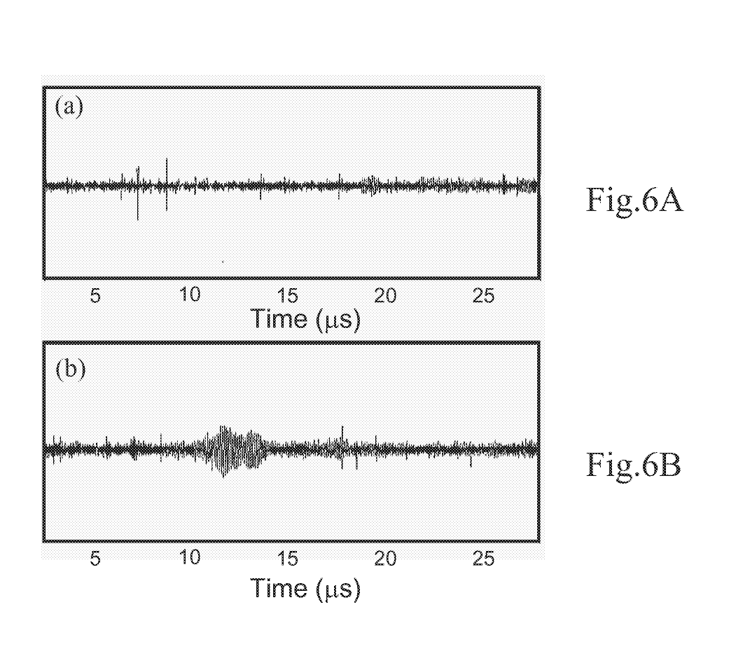

Upon going through the smoothing out process, the smoothed-out signal is sent to the transmit determination system to determine the distance between the detection system transmitting the detection signals and the obstacle reflecting the signal. In the present invention, the subtraction of the reflected signals is used to determine the distance, such that in case after subtraction of the rated values of the two signals, and if the absolute value of the difference is less than 60, then subtract the data of the two points to obtain its slope, since the reflected wave will appear near the angle of 90 degrees, and its frequency of appearance is exceedingly high, therefore, if the absolute value of the slope is between 85 to 90 degrees, then the distance between the detection device and obstacle can be obtained through a counter method to determine the counting point for the reflected signals to appear. Since the period for the reflected signal to cross the zero point is rather fixed, so that it can be used as a way to determine the reflected signal. In the present invention, in order to measure the distance between the detection device and the obstacle more accurately, the absolute value of difference of two adjacent data points is obtained, to display the zero crossing point on the Graphic User Interface, then the position of the obstacle that reflects the detection signal can be determined. FIGS. 6A and 6B are waveforms of the reflected signals on GUI obtained with rib blocking and without rib blocking respectively according to the present invention.

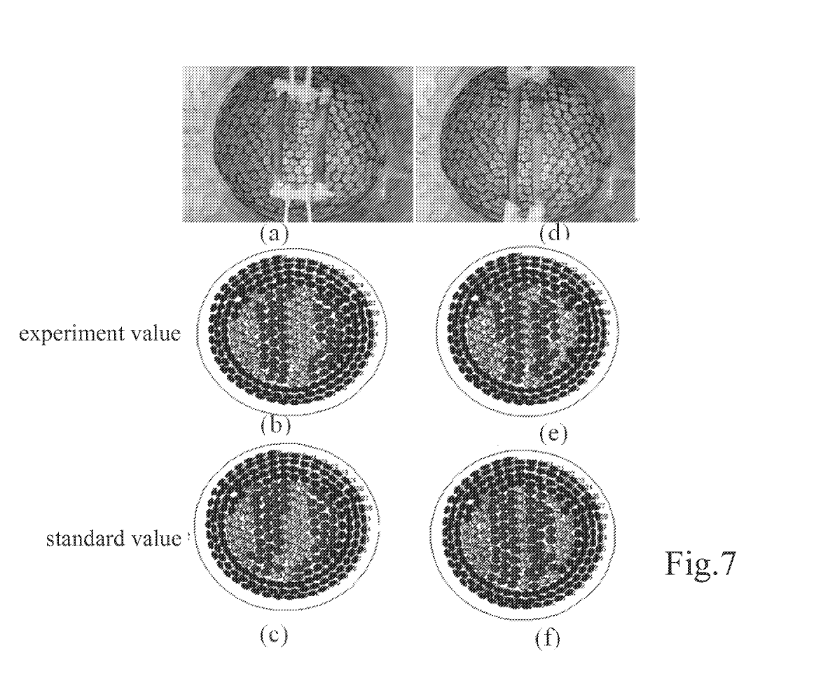

FIGS. 7(a) to 7(f) show the results of tests using different rib pitches, and it determines comparison results with zero crossing distance, to verify the correctness of the zero crossing algorithm of the present invention. Wherein, FIGS. 7(a) and (d) are the results of actual experiments using rib pitches of 1:2 and 1:1. FIGS. 7(b) and 7(c) are the actual measurement result and standard result of FIG. 7(a), the actual measurement result is obtained by means of zero-crossing, and the standard result is obtained by means of hydrophone simulation. FIGS. 7(e) and 7(f) are the actual measurement result and standard result respectively of FIG. 7(d). In FIGS. 7(b), (c),(e),(f), the dark color indicates that rib is not in the detection range, dark gray color indicates detection the presence of rib, and light gray color indicates no presence of rib blocking. According to test results, when the rib pitch is 1:2, the accuracy of standard result and test result is 90%; and when rib pitch is 1:1, the accuracy of standard result and test result is 85%.

Summing up the above, the present invention provides a multi-channel hemispherical ultrasonic wave transmit and receive detection system and method, which switch through each of the channels continuously to receive the ultrasonic waves reflected signals, amplifies the reflected signals through the receive system, and then converts it to digital signals, and transmits them to the Graphic User Interface (GUI) of a computer to proceed with signal analysis, in determining if the ultrasonic wave detection signals hit upon an obstacle, and the distance to the obstacle. The present invention can be used in the rib detection before conducting ultrasonic wave tremor burning therapy, to accurately detect position of rib, and at the same time shut down the probe blocked by the rib, as such solving the problem of rib blocking and ultrasonic wave energy blocking and rib overheating.

The above detailed description of the preferred embodiment is intended to describe more clearly the characteristics and spirit of the present invention. However, the preferred embodiments disclosed above are not intended to be any restrictions to the scope of the present invention. Conversely, its purpose is to include the various changes and equivalent arrangements which are within the scope of the appended claims.

What is claimed is:

1. A multi-channel hemispherical ultrasonic wave transmit and receive detection method, comprising following steps:
transmitting a plurality of single-channel detection signals by means of a transmit control system;
receiving by a receive system through a multiplexer reflected signals of said detection signals which do not transmit through an obstacle and are reflected back;
determining by said receive system a channel of said transmit control system used for said detection signal, to control said multiplexer to switch to said channel, and amplify said detection signals and said reflected signals;
performing high speed analog-to-digital sampling by said receive system for said reflected signals amplified, and then performing high speed transmission of said reflected signals amplified through a USB communication module; and
utilizing a Graphic User Interface (GUI) to receive said reflected signals sent from said receive system via said USB communication module, and to display waveforms of said reflected signals.

2. The multi-channel hemispherical ultrasonic wave transmit and receive detection method as claimed in claim 1, wherein utilizing at least a static random access memory (SRAM) by said receive system to partition said reflected signals sampled into large amount of data, and transmitting them into a field programmable gate array (FPGA), then transmitting said data to said USB communication module via said FPGA.

3. The multi-channel hemispherical ultrasonic wave transmit and receive detection method as claimed in claim 1, wherein before receiving said reflected signals by said hemispherical ultrasonic wave probe, first utilizing a signal amplification and driving system to receive said detection signals sent from said transmit control system, amplifying and then transmitting said detection signals to an energy converter, which sends out said detection signals, then said reflected signals are received by said hemispherical ultrasonic wave probe.

4. The multi-channel hemispherical ultrasonic wave transmit and receive detection method as claimed in claim 1, wherein said multiplexer utilizes an operation amplifier (OPA) to amplify said detection signals and said reflected signals.

5. The multi-channel hemispherical ultrasonic wave transmit and receive detection method as claimed in claim 1, wherein said receive system further includes an analog-to-digital converter, to convert said reflected signal into digital signals at high speed.

6. The multi-channel hemispherical ultrasonic wave transmit and receive detection method as claimed in claim 2, further comprising: utilizing an oscillator to generate a pulse signal, and transmitting it to said field programmable gate array (FPGA).

7. The multi-channel hemispherical ultrasonic wave transmit and receive detection method as claimed in claim 12, wherein upon receiving large amount partitioned data by said FPGA, it transmits said data into a program of said GUI, and said program sets a counter for points representing said received data, and also sets an X axis in a Picture Box; places data received from said USB communication module into an array, and sets a Y axis in said Picture Box, to display waveforms of said reflected signals, and said program utilizes zeroes at end of data as indication of termination.

8. The multi-channel hemispherical ultrasonic wave transmit and receive detection method as claimed in claim 1, Wherein said transmit control system is triggered by a burst wave of 5-period, then it selects a channel to output said burst wave.

9. The multi-channel hemispherical ultrasonic wave transmit and receive detection method as claimed in claim 8, wherein said burst wave triggers said USB communication module at the same time.

10. The multi-channel hemispherical ultrasonic wave transmit and receive detection method as claimed in claim 1, wherein said GUI further performs signal smooth-out processing and noise separating processing for said reflected signals.

11. The multi-channel hemispherical ultrasonic wave transmit and receive detection method as claimed in claim 1, further comprising: utilizing a transmit determination system to determine distance to said obstacle based on waveforms of said reflected signals.

12. The multi-channel hemispherical ultrasonic wave transmit and receive detection method as claimed in claim 11, wherein subtracting by said transmit determination system rated levels of two said reflected signals, and determining distance to said obstacle based on a slope obtained through subtraction of reflected signals.

13. The multi-channel hemispherical ultrasonic wave transmit and receive detection method as claimed in claim 12, further comprising: obtaining an absolute value of subtraction value of two adjacent reflected signals, and displaying a zero-crossing point of a waveform after subtraction on said GUI, then determining distance to said obstacle using said transmit determination system.
